(19) United States
(12) Patent Application Publication
(10) Pub. No.: US 2024/0165322 A1
(43) Pub. Date: May 23, 2024

(54) INTRAVENOUS BAG HOLDER

(71) Applicant: Han-Chun LIN, Taichung City (TW)

(72) Inventor: Han-Chun LIN, Taichung City (TW)

(21) Appl. No.: 17/989,641

(22) Filed: Nov. 17, 2022

(51) Int. Cl. A61M 5/14; F16M 13/04

(52) U.S. Cl. CPC A61M 5/1415; F16M 13/04; A61M 2005/1416

(57) ABSTRACT

An intravenous (IV) bag holder, includes a headwear and at least one fastening members. The headwear is wearable on a user's head and has an external surface including an attaching portion. The fastening member is arranged to span over the attaching portion. With such a structural arrangement, an IV bag can be attached, with an external surface thereof, to the attaching portion, and the fastening member is set on and presses down the IV bag. The attaching portion and the fastening member provide dual constraints that make the IV bag immobile on the headwear or not dropping therefrom. The user may put the headwear on the head easily, so that the holder is convenient for the user to use, and allows for easy observation of a remaining capacity of the IV bag.

### TECHNICAL FIELD OF THE INVENTION

The present invention relates generally to an intravenous (IV) bag holder, and more particularly to a holder that allows an IV bag to be directly combined with and fixed to headwear and ensures that the IV bag does not shift or drop.

### DESCRIPTION OF THE PRIOR ART

Intravenous therapy is a common medical way for treating patients, both inpatients and outpatients, in hospitals, as shown in FIG. 7. Medically, intravenous therapy includes intravenous infusion, in which a venous indwelling needle is manually set up to replenish water, glucose, or electrolyte that the body of the patient may need. Another purpose is to establish an intravenous administration channel to allow medicine to be directly introduced into the blood vessels. Thus, there are just two conditions that requires injection infusion. The first is that the patient cannot take food, or even not drink water, causing dehydration. The second is the patient requires medicine administration through the veins.

A general way of hanging an IV container is an IV pole, such as an IV frame disclosed in Taiwan Utility Model M622983 (which will be referred to “first prior art solution”). The first prior art solution includes an elongate upright pole and a base to which the upright pole is fixed. The base is combined with and fixed to multiple legs that are provided with casters. To use the first prior art solution, for either walking or stepping along a stairway, or just using the toilet, a person has to push and move with such a pole, and must lift up the pole when moving across a doorsill.

This is inconvenient. Further, the noise caused by pushing the pole to move often catches other people's attention. Thus, further improvement is desired.

Taiwan Patent 1480075 discloses method of using balance controlled back-carrying intravenous pole and device thereof (which will be referred to “second prior art solution”). The second prior art solution seemingly resolves the shortcomings of the first prior art solution, yet it develops other drawbacks, such as complicated structure, being hard to wear, requiring other people's help to wear. Considering the situation that a person needs to use the toilet in the midnight, although the restroom is just nearby, the person has to ask other people to help for going through a tedious process to put on the device, and after relieving, a similar complicated process has to be done for taking off the device. This is quite inconvenient, and further improvement is desired.

Taiwan Utility Model M415711 discloses an IV frame cap (which will be referred to “third prior art solution”). The third prior art solution resolves the shortcomings of the first and second prior art solutions and features simple structure and light weight and being easy to wear. However, it arranges an IV frame on a cap, and a highest location of the IV container is spaced from the cap by a distance (which forms a force arm). The IV solution contained in the IV container oscillates with the movement of the wearer's head. The oscillation of the IV solution induces a pushing force that oscillates back and forth, and the wearer's head is uncontrollably dragged by such an oscillating force. To avoid such dragging, the wearer has to release a force from the head to control and hold the IV container in position without being caused to shake due to the oscillation of the IV solution. This makes the head and the neck of the wearer exhausted. Further, due to the distance between the IV container and the cap, a torque is inevitably induced (and such a torque generates an increased centrifugal force), together with the IV solution contained in the IV container having a weight and being flowable, when the wearer bends the head down at an unexpectedly overlarge angle, or the head turns toward the right side or the left side, in addition to the uncomfortable feeling of the head due to being dragged by the centrifugal force, the IV container may undesirably fall from the hat, causing panic and trouble of the wearer. Further, during use, the wearer may feel concerned about being afraid of the falling of the IV container. Thus, the third prior art solution also needs further improvement.

Chinese Patent Application No. 201120576078.x discloses an easy-to-carry self-hating infusion cap (which will be referred to as “fourth prior art solution”). The fourth prior art solution arranges an infusion bag on the top of a cap, and a wide fastening tape is set to cover the infusion bag to prevent the infusion bag from falling. In this way, on the one hand, the hands of the wearer can be freed, and on the other hand, the body temperature may be applied to heat up the infusion bag to reduce the discomfort of the wearer for infusion conducted in a cold weather. Although the fourth prior art solution overcomes some of the problems, it develops other problems. Since the infusion bag is laid flat on the top of the cap, the liquid contained in the infusion bag cannot completely flow out of the bag, so as to affect a normal operation of infusion. Another problem is that the infusion bag is arranged on the top of the cap and is covered by the fastening tape, the wearer or other people may not directly observe the remaining capacity of the infusion bag. The wearer has to first take off the cap before observation can be made to the remaining capacity of the infusion bag. This is quite inconvenient. In case of inadvertence, if it is not timely realized that the liquid of the infusion bag runs out or cannot further flow out, damage may be caused to the body of the wearer. Thus, the fourth prior art solution also need further improvement.

### SUMMARY OF THE INVENTION

In view of the above, the present invention aims to provide a solution that overcomes the numerous drawbacks of the first to fourth prior art solutions.

The primary objective of the present invention is to provide an intravenous (IV) bag holder, which allows a user to put on and take off in a more convenient and easier way and which, when put into use, frees the two hands of the user for doing other activities, and which allows for easy observation of a remaining amount of the IV solution in the IV bag.

Thus, the present invention provides an IV bag holder, which comprises: a headwear and at least one fastening members, wherein:
the headwear is wearable on a user's head and has an external surface that defines a front side surface, a rear side surface, a left side surface, and a right side surface and is extended to a top portion, and the external surface comprises an attaching portion; and the fastening member spans over the attaching portion and comprises a fastening band and a ring buckle having a through hole, the ring buckle being fixed to the headwear and located at one side of the attaching portion, the fastening band having one end fixed to an opposite side of the attaching portion to be opposite, in a transverse direction, to the ring buckle, an opposite end of the fastening band forming a free end, the fastening band comprising a first connecting member and a second connecting member, the free end of the fastening band being extendable through the through hole of the ring buckle to be then folded backward to allow the second connecting member to combine with and fix to the first connecting member.

With the above structural arrangement, an IV bag is attachable, with an external surface thereof, to the attaching portion. By means of an electrostatic attraction effect, or a frictional resisting force, or connection with a hook-and-loop fastener between the IV bag and the attaching portion, the IV bag is prevented from planar movement relative to the attaching portion. The fastening band is then extended through the through hole of the ring buckle and is folded back to have the fastening band tightly positioned on the IV bag and the second connecting member combined with and fixed to the first connecting member, so as to have the IV bag combined with and fixed to the headwear, and the IV bag is constrained in each of frontward, rearward, leftward, rightward, upward, and downward directions and is thus immobile. A user, when putting the headwear on the head, is free of concern about shifting or falling of the IV bag, and the IV bag can be snugly positioned on the external surface of the headwear, at a location close to the top portion by following the contour of the head, so that the weight of the IV bag is spread and does not concentrate at one side. In this way, when the patient is going out or using the toilet, it is convenient and easy to carry the IV bag and can free the two hands of the user for doing other activities. There is no need for hanging the IV bag with a pole that has a complicated structure and is heavy.

### BRIEF DESCRIPTION OF THE DRAWINGS

FIG. 1 is a side elevational view showing a first embodiment of the present invention.

FIG. 1A is a schematic view showing an intravenous (IV) bag according to the present invention.

FIG. 2 is a side elevational view showing the first embodiment of the present invention to which an intravenous bag is combined and fixed.

FIG. 3 is a schematic view showing the first embodiment of the present invention worn on a user's head.

FIG. 4 is rear view of the present invention.

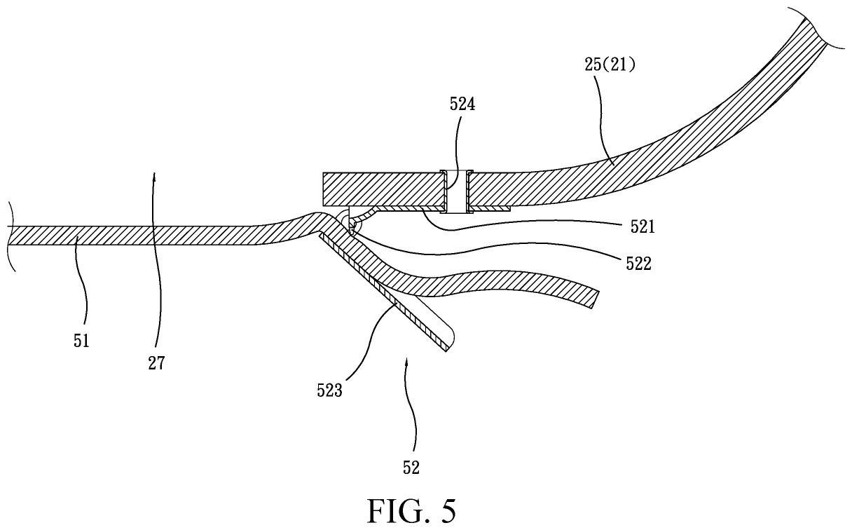

FIGS. 5 and 6 are cross-sectional views taken along line A-A′ of FIG. 4, demonstrating an operation thereof.

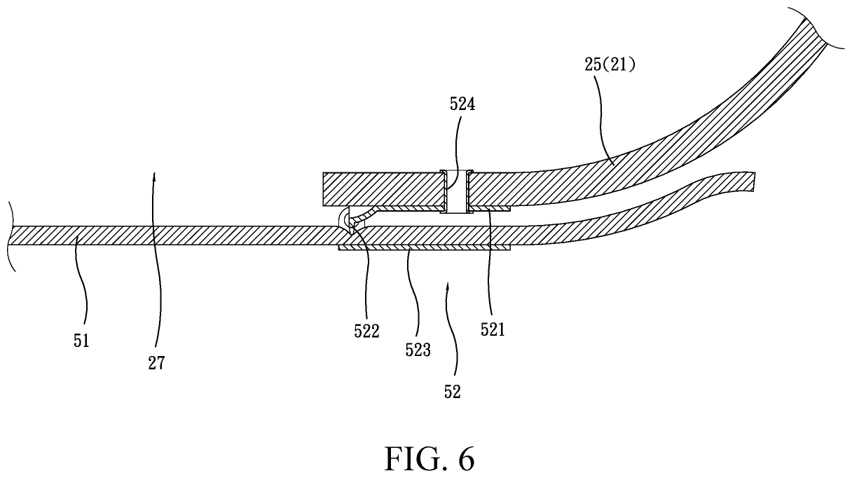

FIG. 7 is a schematic view demonstrating use of an IV pole hanging an IV bag known in the prior art.

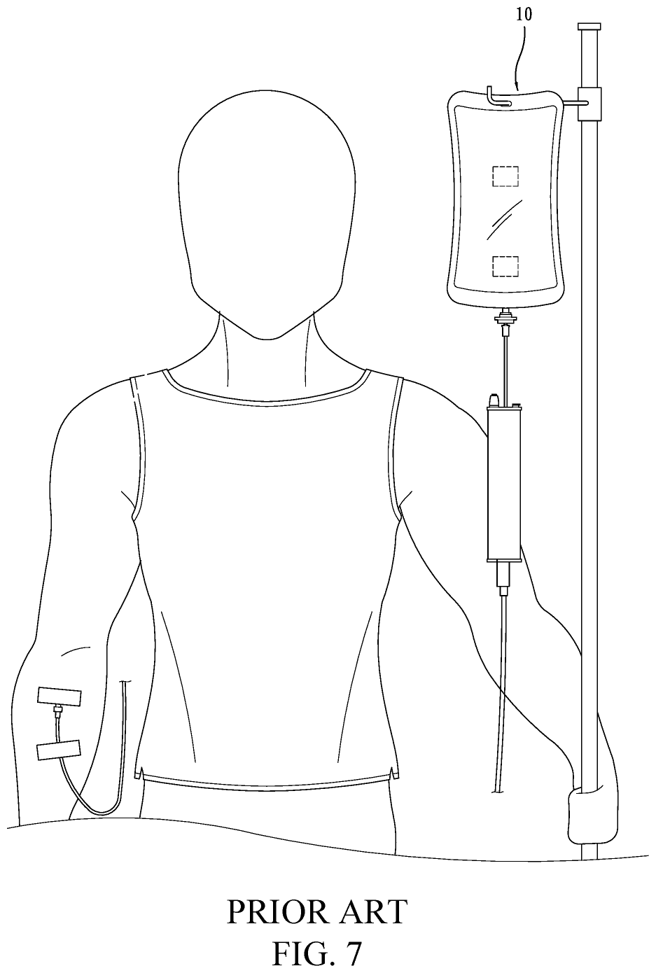

### DETAILED DESCRIPTION OF THE PREFERRED EMBODIMENT

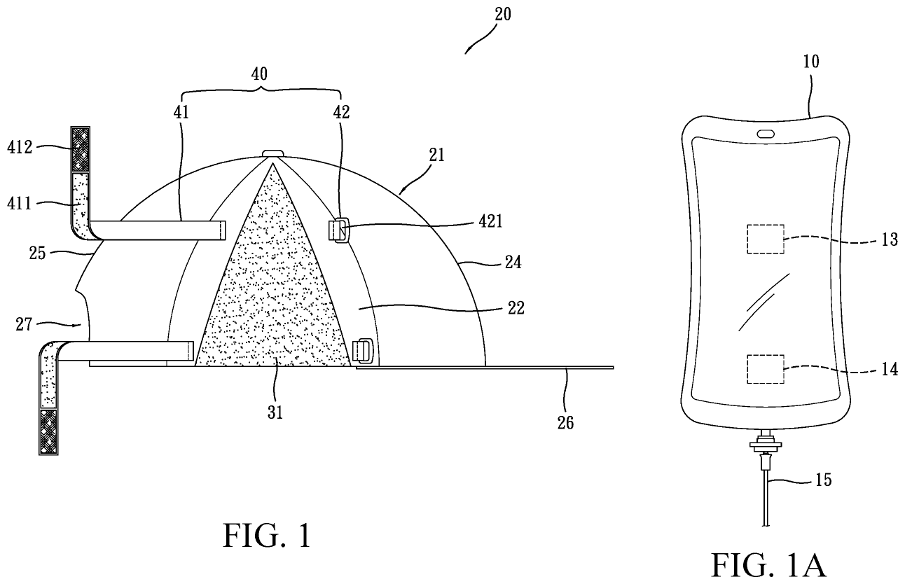

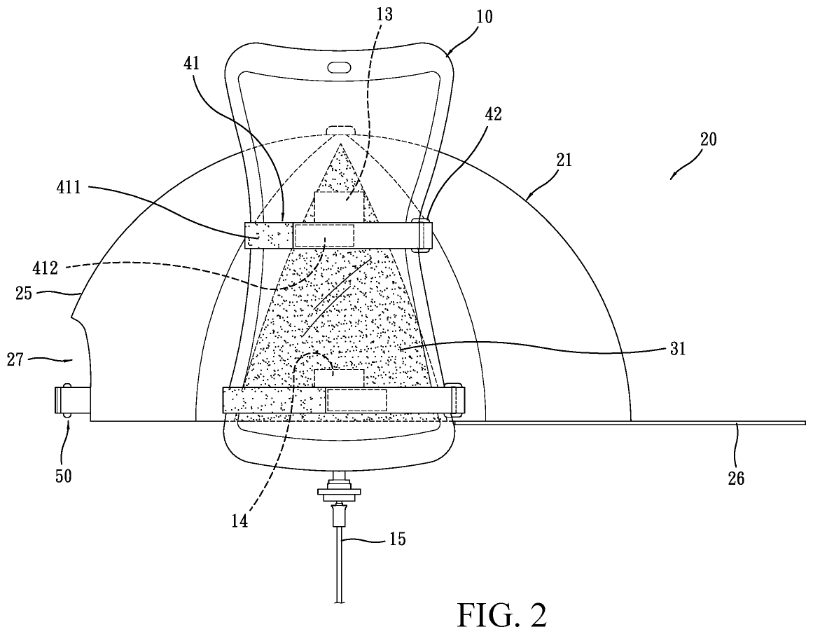

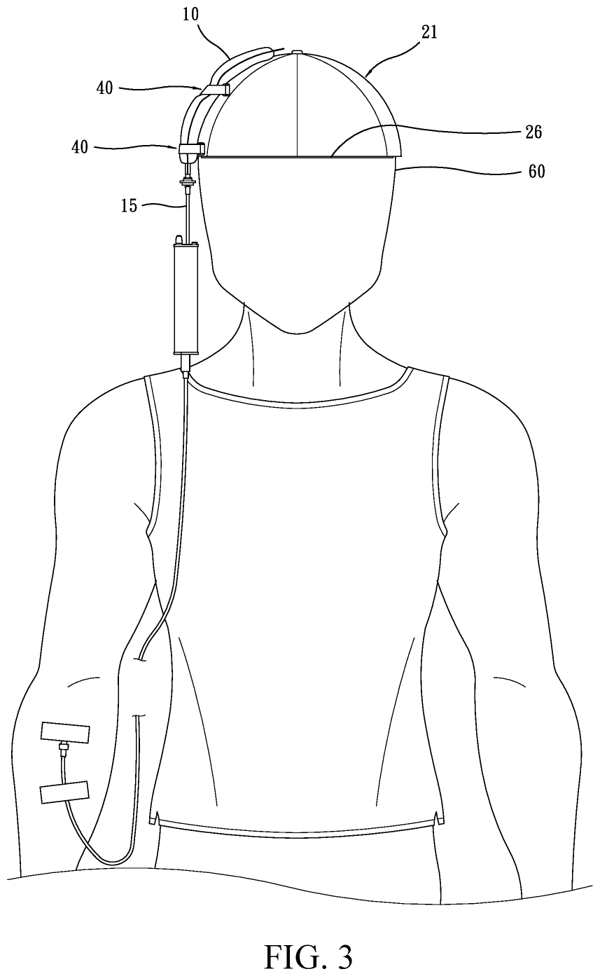

Firstly, referring to FIGS. 1-3 and, also, FIG. 1A, the instant embodiment provides an intravenous (IV) bag holder 20, which comprises a headwear 21, two fastening members 40, and an adjusting member 80.

The headwear 21 is wearable on a user's head 60 and has an external surface that defines a front side surface 24, a rear side surface 25, a left side surface 23, and a right side surface 22 and extended to a top portion. The front side surface 24 is provided with a projecting gripping portion 26. The gripping portion 26 has a moderate level of stiffness and is provided for being gripped and held by the user's hand. The rear side surface 25 is formed with an opening 27, and the opening 27 is extended to a bottom edge of the headwear 21. The headwear 21 is made of cotton fabric or plastic fiber fabric.

The external surface of the headwear 21 is provided with an attaching portion. The attaching portion 31 is provided for receiving an IV bag 10 to attach thereto, such that the IV bag 10 is fixed in position, and thus immobile, on the attaching portion 31. In the instant embodiment, the attaching portion comprises a hook-and-loop fastener; however, the attaching portion 31 can alternatively be an electrostatic patch that is combined with and fixed to the headwear or a patch of which a surface is coated with a plastic/rubber-like substance layer, or is formed by directly coating a plastic/rubber-like substance layer on the headwear 21. In the case that the attaching portion 31 includes a hook-and-loop fastener or an electrostatic patch or a patch having a surface coated with a plastic/rubber-like substance layer, the combination thereof with the headwear 21 can be bonding with adhesive, or fixing with sewing, or riveting, or fastening with a button, or fastening with a compression buckle, and the way of combination thereof with the headwear 21 is not limited. The plastic/rubber-like substance layer mentioned above includes one of plastics, rubber, silicone, and latex. The instant embodiment arranges the attaching portion 31 on the right side surface 22 of the headwear, and in fact, it can be arranged on the left side surface 23. The attaching portion 31 has a width and a height, and is extended upward from the bottom edge of the headwear 21 to approach the top portion of the headwear 21.

The fastening members 40 are arranged to span across above the attaching portion 31. The two fastening members 40 are arranged to have a spacing distance therebetween and shows a relationship of being located at upper and lower positions. Each of the fastening members 40 comprises a fastening band 41 and a ring buckle 42 having a through hole 421. The ring buckles 42 are each fixed to the headwear 21 and are set the same side of the attaching portion 31. Each of the fastening bands 41 has an end fixed to an opposite of the attaching portion 31 to be opposite, in a transverse direction, to one of the ring buckles 42. Each of the fastening bands 41 has an opposite end that forms a free end. Each of the fastening band 41 is provided with a first connecting member 411 and a second connecting member 412. The second connecting member 412 is arranged at an end portion associated with the free end of the fastening band 41. The first connecting member 411 and the second connecting member 412 are both located on an outside surface of the fastening band 41. The free end of each of the fastening bands 41 is arranged to extend through the through hole 421 of the corresponding ring buckle 42 and is then folded back to allow the second connecting member 412 to combine with and fix to the first connecting member. In the instant embodiment, the first connecting member 411 and the second connecting member 412 components of a hook-and-loop fastener that are combinable with and fixed to each other.

However, the combination between the first connecting member 411 and the second connecting member 412 can be realized with other known measures, such a button, a compression buckle, and a hook. Further, it is also feasible to include just one single fastening member 40, and in case that a single fastening member 40 is adopted, widths of the fastening band 41 and the ring buckle 42 may be enlarged.

The adjusting member 50 spans between two ends of the opening 27 of the headwear 21 and comprises a strip 51 and a buckle 52. The buckle 52 comprises a fixed plate 521 and a movable plate 523. An end of the movable plate 523 is pivotally connected to the fixed plate 521, so that the movable plate 523 is movable, through pivoting, on the fixed plate 521. The fixed plate 521 has an end that is combined with and fixed to the headwear 21 by a rivet 524 to be located at one end of the opening 27 and an opposite end that is formed with teeth 522 raised upward therefrom. The strip 51 has an end combined with and fixed to the headwear 21 at another end of the opening 27, and the strip 51 has an opposite end that is extendable through between the fixed plate 521 and the movable plate 523, and one surface of the strip 51 faces the teeth 522. When the movable plate 523 is. Pressed down, the strip 51 is set in tight abutting engagement with the teeth 522 so as to make the strip 51 immobile. When the movable plate 523 is lifted upward, the tight abutting engagement between the strip 51 and the teeth 522 is released, and the strip 51 resumes movable. When the strip 51 is shifted rightward, the opening 27 is reduced, and under such a condition, the headwear 21 becomes tight in wearing, and when the strip 51 is shifted leftward, the opening 27 is enlarged, and under such a condition, the headwear 21 becomes loose in wearing.

Thus, the above provides a description for each of the components of the IV bag holder 20 according to the preferred embodiment of the present invention and the assembly thereof. In the following, features of use will be described.

Continuously referring to FIGS. 1-3 and, also, FIG. 1A, when a person gets sick and becomes an inpatient to be treated with intravenous therapy, one or multiple hook-and-loop fasteners 13, 14 are first attached to an external surface of an IV bag 10 supplied by the hospital, and in the instant embodiment, the attaching portion 31 is also a hook-and-loop fastener, so that the hook-and-loop fasteners 13, 14 attached on the IV bag 10 are attached to and combined with the attaching portion 31, so that the IV bag 10 is constrained from moving in either front, rear, left, or right directions. Then, the two fastening bands 41 are each extended through the through hole 421 of the ring buckle 42 corresponding thereto and then folded back and make the fastening band 41 pressed toward and placed tightly on the IV bag 10, and making the second connecting member 412 combined with and fixed to the first connecting member 411. Then, the user may grip the gripping portion 26 with the hands to put the headwear 21 combined with the IV bag 10 on the head 60. Since the gripping portion 26 has a proper level of stiffness, it provides a sufficient gripping force and supporting force to have the headwear 21 and the IV bag 10 moving together. After the headwear 21 has been put on the head 60, adjustment is made for the adjusting member 50 to provide a proper level of tightness between the headwear 21 and the head 60. Since the IV bag 10 is subjected to dual constraints by the attaching portion 31 and the two fastening members 40 from moving in each of upward, downward, leftward, and rightward directions and being prevented from oscillating, there will be of no concern about the IV bag 10 falling from the headwear 21.

It is additionally noted here that when the attaching portion 31 is formed of the electrostatic patch or a patch having a surface coated with a plastic/rubber-like substance layer combined with and fixed to the headwear 21, or the attaching portion 31 is formed by directly coating a plastic/rubber-like substance layer on the external surface of the headwear, there is no need to provide the hook-and-loop fasteners 13, 14 on the external surface of the IV bag 10. Since the material of the IV bag 10 is either polyvinyl chloride (PVC) or polypropylene (PP), the IV bag 10 is attached to the electrostatic patch, an electrostatic attracting effect between the two prevents the IV bag from planar movement; and when the IV bag 10 is attached to a patch having a surface coated with a plastic/rubber-like substance layer or a plastic/rubber-like substance layer provided on the headwear 21, a frictional resisting force between the two similarly prevent the IV bag 10 from planar movement; and when the two fastening members are set to press against the IV bag 10, the IV bag 10 is securely fixed on the headwear without shifting or dropping.

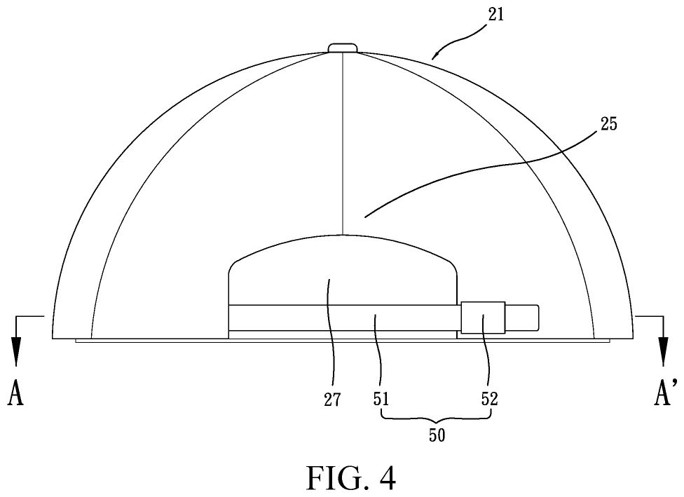

Further referring to FIG. 3, since the IV bag 10 and the IV solution contained therein are soft and shape-changeable, when the headwear 21 is put on the user's head 60, the upper end of the IV bag 10 naturally curves to become snugly close to the top portion of the headwear 21. Since there is almost no force arm formed between the IV bag 10 and the headwear 21, no large centrifugal effect may be induced when the user's head is moving, and the IV solution in the IV bag 10 is divided into three regions as being compressed by the two fastening bands 41, so that the flowability of the IV solution in the IV bag 10 is constrained, and the IV solution no longer generates a large oscillating force when the IV bag 10 is being shaken. Further, the gravity of the IV solution is divided and spread, and does not concentrate at the right side of the user's head 60. It is known that the IV bag has two common specifications, which are 500 ml and 1000 ml. The 500 ml IV bag is the most commonly used one, and the weight of a 500 ml IV bag is around 0.5 Kg. When the IV bag 10 is divided into three regions, a vertical force that actually acts on the right side of the headwear 21 is greatly reduced. Although the user may perceive some gravitational force induced on the right side of the head 60, such a gravitational force can be readily born by an ordinary person. Further, there will be no situation of the headwear 21 being inclined. And, with the consumption of the IV solution, the IV bag becomes lighter and lighter. It is particularly noted here that the IV bag is not set horizontal on the headwear 21 and is attached to the headwear 21 at an inclination angle relative to a vertical direction, so that a dispensing tube 15 of the IV bag 10 is located on a lower side. Thus, there will be no situation that the IV solution cannot flow downward, and a remaining amount of the IV solution in the IV bag 10 can be easily observed.

Based on the above description, it is appreciated that the IV bag holder according to the present invention can be fast put on and taken off just like a normal way of wearing a hat. When a person is sicks and stays in a hospital, for 24 hours a day, either going out for a short period, or staying outside for a long period of time, or simply using the toilet, the headwear 21 can be easily worn and removed. The IV bag 10 is combined with and fixed to the headwear 21, and it needs just one time of assembling. When the user lies down on a sick bed, there is no need to detach and separate the IV bag 10 from the headwear 21, and instead, the IV bag 10 is hung, together with the headwear 21, on an existing IV pole supplied by the hospital. Each time of using the toilet or going out, it only needs to put on the headwear 21. This is extremely convenient for use and may free the hands of the user for doing other activities.

It is appreciated from the above description, the structure of the present invention is simple, and the manufacturing cost is low. Through efficient wearing and removing the headwear 21, in combination with the attaching portion 31 arranged in a longitudinal direction and the fastening members 40 arranged in a transverse direction, the IV bag 10 is securely combined with and fixed to the headwear 21, and the fastening members 40 divide, through compression, the IV bag 10 into multiple regions, so that the flowability of the IV solution in the IV bag 10 is constrained, without causing an excessive oscillating force to drag the user's head 60 or body. Further, the flexibility and softness of the IV bag 10 and the IV solution allow for snugly positioning on the headwear 21 to spread the force born by the head 60 and also to greatly reduce the vertical force on one side of the IV bag 10 to thereby prevent the headwear 21 from inclining toward one side. The structure of the present invention is simple, yet bringing great convenience and consumer-friendly cost to the patients, and benefiting all the patients on Earth.

I claim:

1. An intravenous (IV) bag holder, comprising a headwear and at least one fastening member, wherein:
the headwear is adapted to be put on a head of a user and has an external surface that comprises an attaching portion, the external surface defining a front side surface, a rear side surface, a left side surface, and a right side surface and extended to a top portion; and
the fastening member spans over the attaching portion and comprises a fastening band and a ring buckle having a through hole, the ring buckle being fixed to the headwear and located at one side of the attaching portion, the fastening band having one end fixed to an opposite side of the attaching portion to be opposite, in a transverse direction, to the ring buckle, an opposite end of the fastening band forming a free end, the fastening band comprising a first connecting member and a second connecting member, the free end of the fastening band being extendable through the through hole of the ring buckle to be then folded backward to allow the second connecting member to combine with and fix to the first connecting member;
wherein dual constraints achieved with the attaching portion and the fastening member are adapted to allow an IV bag to combine with and fix to the headwear.

2. The IV bag holder according to claim 1, wherein the attaching portion comprises one of a hook-and-loop fastener, an electrostatic patch, and a patch having a surface coated with a plastic/rubber-like substance layer and is combined with and fixed to the headwear.

3. The V bag holder according to claim 1, wherein the attaching portion comprises a plastic/rubber-like substance layer coated on the external surface of the headwear.

4. The IV bag holder according to claim 1, wherein the front side surface of the headwear further comprises a gripping portion combined with the headwear, the gripping portion having stiffness.

5. The IV bag holder according to claim 1, wherein the headwear further comprises an adjusting member, and the rear side surface of the headwear is formed with an opening, wherein the adjusting member is arranged to span between two ends of the opening and comprises a strip and a buckle, the buckle comprising a fixed plate and a movable plate, an end of the movable plate being pivotally mounted to the fixed plate so that the movable plate is movable, through rotation, on the fixed plate, the fixed plate having an end combined with and fixed to the headwear and located at one of the ends of the opening and an opposite end that is provided with teeth raised upward therefrom, one end of the strip being combined with and fixed to the headwear at another one of the ends of the opening, an opposite end of the strip being extendable through between the fixed plate and the movable plate, one surface of the strip facing the teeth, so that when the movable plate is pressed downward for closing, the strip is set in tight abutting engagement with the teeth, making the strip immobile, and when the movable plate is lifted upward, the tight abutting engagement between the strip and the teeth is released, and the strip resumes movable for adjusting tightness of the headwear.

6. The IV bag holder according to claim 1, wherein the at least one fastening member comprises multiple fastening members that are arranged, in a relationship of being at upper and lower positions and spaced from each other to span over the attaching portion.

7. The IV bag holder according to claim 1, wherein the first connecting member and the second connecting member comprise hook-and-loop fasteners that are combinable with and fixable to each other.

8. The IV bag holder according to claim 1, further comprising an IV bag, the IV bag having an external surface on which one or multiple hook-and-loop fasteners are attached, the attaching portion comprising a hook-and-loop fastener, the hook-and-loop fasteners of the IV bag being combinable with and fixable to the hook-and-loop fastener provided on the headwear.

9. The IV bag holder according to claim 2, wherein the plastic/rubber-like substance layer comprises one of plastics, rubber, silicone, and latex.

10. The IV bag holder according to claim 3, wherein the plastic/rubber-like substance layer comprises one of plastics, rubber, silicone, and latex.
